(19) United States
(12) Patent Application Publication
(10) Pub. No.: US 2019/0071687 A1
(43) Pub. Date: Mar. 7, 2019

(54) A METHOD FOR INCREASING RESISTANT STARCH AND DIETARY FIBER IN RICE

(71) Applicants: UDAYA AGRO FARM, Kumbakonam (IN); GREEN FLORA BIOSCIENCES PVT. LTD, Chennai (IN)

(72) Inventor: R. Bharathi Raja, Tindivanam (IN)

(21) Appl. No.: 16/077,670

(22) PCT Filed: Feb. 2, 2017
(86) PCT No.: PCT/IB2017/050558

§ 371 Date: Aug. 13, 2018

(30) Foreign Application Priority Data
Feb. 15, 2016 (IN) 201641005181

(51) Int. Cl. C12N 15/82; C12N 9/10; A01H 6/46

(52) U.S. Cl. CPC C12N 15/8245; C12Y 204/01011; A01H 6/4636; C12N 9/1051

(57) ABSTRACT

The present invention discloses mutations in the genes encoding starch synthases and also in starch branching enzymes associated with enhanced dietary fibre and resistant starch levels in the endosperm of a suitable variety of rice. The dietary fiber and resistant starch are enhanced to an extent to significantly reduce the hydrolysis index values of the rice grains to 35%-40%. These rice varieties are in great demand for diabetic population and provide a number of other health benefits such as reduced body weight gain, cardiac health and colon health. As this strategy does not involve the use of genetic manipulation technologies, it can be directly employed in the rice breeding programmers without any restrictions.

### TECHNICAL FIELD OF THE INVENTION

The present invention relates to a rice grain obtained from a mutant rice plant with increased dietary fiber and resistant starch expression. More particularly, the invention relates to a method of chemically induced double or triple mutations in the genes encoding starch synthases (ssI and/or ssIIIa) in combination with mutations in genes encoding starch branching enzymes (sbeI and/or sbeIIb) of rice, leading to modification of amylopectin structure, which results in increased resistant starch and dietary fibre contents and thereby reduces the hydrolysis index.

### BACKGROUND OF THE INVENTION

Cereal grains such as rice are basic food components of the human diet and contain important nutrients such as dietary fibre and carbohydrates. The consumption of dietary fibre is particularly important for digestion and has been implicated as being useful for the prevention or treatment of certain diseases such as diabetes, obesity and colon cancer. Generally, dietary fibre is defined to be remnants of plant materials that are resistant to digestion by human alimentary enzymes, including non-starch polysaccharides, resistant starch, lignin and minor components such as waxes, cutin and suberin. Because of the potential health benefits of foods rich in dietary fibre, many countries have recommended the increased consumption of such foods as a part of their dietary guidelines.

White rice is a dietary staple for more than half the world's population. A new study from the Harvard School of Public Health shows that the people who consume white rice regularly may significantly raise their risk of developing type 2 diabetes. They also found that people who consumed rice were more than 1.5 times likely to have diabetes than people who ate the least amount of rice. What's more serious outcome of the study is that for every 5.5 ounces-serving of white rice a person consumed each day, the risk rose by 10 percent. “Asian countries are at a higher risk,” the researchers wrote in the study, published in the March 2015 issue of the British Medical Journal.

White rice is a highly refined staple cereal, which is devoid of almost all fibres and minerals. Major portion of the fibre and minerals are present in the bran layer of rice, which is completely removed by the modern rice milling and polishing machineries. It has been a common practice in the modern rice mills to adopt a high degree of polishing as the consumers prefer well-polished rice due to its better palatability than an unpolished or partly polished grain of rice. In the context of the issue of dilemma between health and palatability, rice eating populations around the globe are looking for an option in which both the issues are being positively addressed.

Diabetes mellitus generally known as diabetes is the most common endocrine disorder in both the developing and the developed nations. Diabetes is a chronic disease, which occurs when the pancreas fails to produce enough insulin, or when the body is not able to effectively use the insulin it produces. This leads to an increased concentration of glucose in the blood (hyperglycemia). Type 1 diabetes previously known as insulin-dependent or childhood-onset diabetes is characterized by lack of insulin production whereas, Type 2 diabetes formerly called non-insulin-dependent or adult-onset diabetes is caused by the body's inability to use insulin effectively. This happens due to excessive body weight and physical inactivity. Another type of diabetes, termed as gestational diabetes, is hyperglycemia, which is first recognized during pregnancy.

Planning and achieving a proper diet for diabetic patients is the mainstay in clinical strategy of the diabetes management. As carbohydrates form the major fraction of food and an indispensable causal factor for glucose release, current dietary diabetes management strategies focus on altering the carbohydrate metabolism in humans to achieve slow release of glucose into the blood stream. This strategy warrants alterations in carbohydrate chemistry and composition in food stuffs to make them medically acceptable to manage diabetes.

The Glycemic Index (GI) is a ranking of carbohydrates based on their immediate effect on blood glucose levels. Foods that raise blood sugar content quickly, have high GI values. Conversely, foods that raise blood sugar content slowly have low GI values. As a result, the GI is useful indicator of starch digestion of food-based products. World health organization define GI as the incremental area under the blood glucose response curve of a 50 g available carbohydrate portion of a test food, expressed as a percent of the response to the same amount of carbohydrate from a standard food consumed by the same subject. The GI consists of a scale from 1 to 100, indicating the rate at which 50 grams of carbohydrate in a particular food is absorbed into the bloodstream as blood-sugar. Glucose itself is used as the main reference point and is rated 100. The GI values of foods are grouped into low GI (<55), medium (55-70), and high (>70) (Miller et al, 1992). During digestion, carbohydrates that break down quickly have high GI. On the other hand, carbohydrates that break down slowly have low GI. Lowering postprandial blood glucose by consuming low GI foods has positive health outcomes for both healthy subjects and patients with insulin resistance.

Cooked rice is readily digested because it contains a higher percentage of digestible starch (DS) and a lower percentage of resistant starch (RS), as a result rice is not the fittest food in the nutritional and medical terms. As it is known fact that rice possesses relatively high glycemic response compared with other starchy foods. High starch and low non-starch polysaccharide contents of polished rice means that rice typically gives a high glycemic response and contain low levels of dietary fiber and resistant starch. Jenkins et al. (1981) reported a very high GI value of 83 for white rice. Many other studies carried out with more number of rice varieties also indicated its high GI status.

Hence, to address the problem of high GI of rice, the viable solution is to increase the fraction of dietary fibre and resistant starch (RS) in rice plants. Dietary fibre and RS elicits three major effects when included in the diet that is dilution of dietary metabolizable energy, a bulking effect and fermentation to short-chain fatty acids and increase in the expression of Peptide YY (PYY) and glucagon-like peptide (GLP)-1 in the gut. RS that has physiologic effects similar to fibre is of utmost importance in rice based diet. Understanding the genetic control of dietary fibre and RS accumulation in rice is of utmost importance for enhancing its nutritional quality. Research on dietary fibre and RS contents in rice assumes considerable significance given the dramatic increase in the incidence of type II diabetes and colorectal cancer in South East Asian countries that are increasingly adopting western diets.

Hence, looking at the problems that exist in the current state of the art, it is desirable to produce rice that has characteristics such as high dietary fiber, resistant starch and low glycemic index.

### SUMMARY OF THE INVENTION

Looking at the problems that exist in current state of the art, it is desirable to make use of induced mutations in the key candidate genes that modify amylopectin structure, which in turn results in the enhancement of resistant starch and dietary fiber in the grains of rice plant.

The present invention describes the method of induced double or triple mutations in genes encoding different starch synthases (ssI and/or ssIII) in combination with starch branching enzymes (sbeI and/or sbeIIb) of suitable rice varieties. These mutations are associated with down-regulation of those key enzymes in grain starch biosynthesis. Down regulation of such target enzymes leads to increased resistant starch and dietary fibre accumulation in rice grains. The increased dietary fibre and resistant starch brings down the hydrolysis index (HI) to very low levels of 35%-40% as compared to the wild type rice variety (control) with HI of 72.6%. HI is an in vitro predicted equivalent indicator of Glycemic Index (GI) of any food.

In accordance with one or more embodiments, the present invention describes double and triple rice mutants harboring mutations in two different gene families namely, mutations in genes encoding one or more starch synthases (SSI and/or SSIIIa) in combination with mutations in genes encoding one or more starch branching enzymes (sbeI and/or sbeIIb) of a suitable rice variety subjected to mutagenesis and further selection by a genomics assisted mutation screening method called as Targeting Induced Local Lesions IN Genomes (TILLING) by sequencing. The mutation is performed by treatment of seeds in a suitable rice variety with a mutagen that is Ethyl Methane Sulfonate (EMS) or N=N=nitroso methyl urea (NMU). As mutagenesis is a random event, various mutants are produced by the mutagenic treatment and the mutant population is then subjected to TILLING by sequencing to screen double or triple mutations in the two gene families namely Starch Synthases and Starch Branching enzymes. These mutations are functionally validated through bioinformatics pipelines SIFT and proven for their role in down regulation of a combination of Starch Synthase and Starch Branching Enzymes, which leads to increased dietary fibre and resistant starch accumulation in rice grains.

The invention is employed to enhance the total dietary fibre from 7% to 13% along with resistant starch content from 5% to 12% in any variety of rice. These desirable features reduced the glycemic response factor namely hydrolysis index of rice grains hence making rice suitable for diabetics. In addition, high dietary fibre content provides a number of health benefits such as reduced body weight, cardiac health and colon health etc. Hence, these mutant rice varieties serve as a healthy alternative cereal staple for general public as well.

### BRIEF DESCRIPTION OF THE DRAWINGS

The foregoing and other features of embodiments will become more apparent from the following detailed description of embodiments when read in conjunction with the accompanying drawings. In the drawings, like reference numerals refer to like elements.

FIG. 1 shows a table depicting amylopectin chain distribution, amylose content, resistant starch content, total dietary fibre and hydrolysis index in the grains of the rice mutant lines Lotus 1-4 and wild type GFRL 78, in accordance to one or more embodiments of the invention.

FIG. 2 shows a flow chart, which explains the work flow employed to isolate mutants in two gene families namely Starch Synthases and Starch Branching enzymes with potential for enhanced resistant starch and total dietary fibre expression in rice grains in accordance to one or more embodiment of the present invention.

FIG. 3 shows a chromatogram generated through Fluorescence Assisted Capillary Electrophoresis (FACE) depicting the amylopectin chain length of Lotus 1 mutant in accordance to one or more embodiment of the present invention.

FIG. 4 shows a chromatogram generated through Fluorescence Assisted Capillary Electrophoresis (FACE) depicting the amylopectin chain length of Lotus 2 mutant in accordance to one or more embodiment of the present invention.

FIG. 5 shows a chromatogram generated through Fluorescence Assisted Capillary Electrophoresis (FACE) depicting the amylopectin chain length of Lotus 3 mutant in accordance to one or more embodiment of the present invention.

FIG. 6 shows a chromatogram generated through Fluorescence Assisted Capillary Electrophoresis (FACE) depicting the amylopectin chain length of Lotus 4 mutant in accordance to one or more embodiment of the present invention.

FIG. 7 shows a chromatogram generated through Fluorescence Assisted Capillary Electrophoresis (FACE) depicting the amylopectin chain length of wild type variety GFRL 78 in accordance to one or more embodiment of the present invention.

FIG. 8 shows a graph of amylopectin chain length distribution of Lotus 1 mutant as compared to wild type GFRL 78 in accordance to one or more embodiment of the present invention.

FIG. 9 shows a graph of amylopectin chain length distribution of Lotus 2 mutant as compared to wild type GFRL 78 in accordance to one or more embodiment of the present invention.

FIG. 10 shows a graph of amylopectin chain length distribution of Lotus 3 mutant as compared to wild type GFRL 78 in accordance to one or more embodiment of the present invention.

FIG. 11 shows a graph of amylopectin chain length distribution of Lotus 4 mutant as compared to wild type GFRL 78 in accordance to one or more embodiment of the present invention.

FIG. 12 shows a table depicting the list of mutations identified in the key candidate genes of mutants Lotus 1-4, leading to increase in the dietary fiber and resistant starch contents in the rice plant, in accordance to one or more embodiments of the invention.

FIG. 13 shows a table depicting the list of alterations in amino acid sequences observed in the mutants Lotus 1-4 and their bioinformatic validation with reference to wild type protein, in accordance to one or more embodiments of the invention.

FIG. 14 shows cDNA sequence of Starch Synthase I gene along with the mutation, in accordance to one or more embodiments of the invention.

FIG. 15 shows protein sequence of Starch Synthase I along with the altered amino acid, in accordance to one or more embodiments of the invention.

FIG. 16 shows DNA sequence of the gene coding Starch Synthase I along with the mutation, in accordance to one or more embodiments of the invention.

FIG. 17 shows cDNA sequence of Starch Synthase IIIa gene along with the mutation, in accordance to one or more embodiments of the invention.

FIG. 18 shows protein sequence of Starch Synthase IIIa along with the altered amino acid, in accordance to one or more embodiments of the invention.

FIG. 19 shows DNA sequence of gene coding Starch Synthase IIIa along with the mutation, in accordance to one or more embodiments of the invention.

FIG. 20 shows cDNA sequence of Starch Branching enzyme I gene along with the mutation, in accordance to one or more embodiments of the invention.

FIG. 21 shows protein sequence of Starch Branching enzyme I along with the altered amino acid, in accordance to one or more embodiments of the invention.

FIG. 22 shows DNA sequence of gene encoding Starch Branching Enzyme I along with the mutation, in accordance to one or more embodiments of the invention.

FIG. 23 shows cDNA sequence of Starch Branching Enzyme IIb gene along with the mutation, in accordance to one or more embodiments of the invention.

FIG. 24 shows protein sequence of Starch Branching Enzyme IIb along with the altered amino acid, in accordance to one or more embodiments of the invention.

FIG. 25 shows DNA sequence of the gene encoding Starch Branching Enzyme IIb along with the mutation, in accordance to one or more embodiments of the invention.

FIG. 26 shows thermographs generated through Differential Scanning Calorimeter (DSC) depicting the gelatinization temperature of starch of rice flour from the wild type rice variety GFRL 1 (a) and mutants Lotus 1 to 4 (b to e).

FIG. 27 shows viscosity graphs generated through Rapid Visco Analyser (RVA) depicting the pasting properties of starch in rice flour samples at different temperature regimes from the wild type rice variety GFRL 1 (a) and mutants Lotus 1 to 4 (b to e).

FIG. 28 shows distribution of granule sizes of rice starch measured through a particle size analyzer of the wild type rice variety GFRL 1 and mutants Lotus 1 to 4.

### DETAILED DESCRIPTION OF THE INVENTION

Reference will now be made in detail to the description of the present subject matter, one or more examples of which are shown in figures. Each example is provided to explain the subject matter and not a limitation. Various changes and modifications obvious to one skilled in the art to which the invention pertains are deemed to be within the spirit, scope and contemplation of the invention.

In order to more clearly and concisely describe and point out the subject matter of the claimed invention, the following definitions are provided for specific terms, which are used in the following written description.

The term “Resistant starch”, means portion of the starch, which is not broken down by human enzymes in the small intestine. It enters the large intestine where it is partially or wholly fermented, as context requires.

The term “Mutation”, means a permanent heritable change in the DNA sequence of a gene that can alter the amino acid sequence of the protein encoded by the gene, as context requires.

The term “Glycemic index”, we mean a numerical scale used to indicate how fast and how high a particular food can raise the blood glucose (blood sugar) level, as the context requires.

The term “Hydrolysis index”, means an in vitro laboratory method to predict Glycemic index of a food stuff, as context requires.

The present invention overcomes the drawback of the existing state of the art technologies by exhibiting mutations in combinations in two major key target gene families starch synthases and starch branching enzymes that are involved in starch biosynthesis. These mutations in combination modifies the amylopectin structure there by leading to increase in dietary fiber (DF) and resistant starch (RS) contents in the rice grains. The above methodology is successful in enhancing the dietary fibre and resistant starch levels to very high levels to significantly reduce the hydrolysis index (HI) values to 33%-40%.

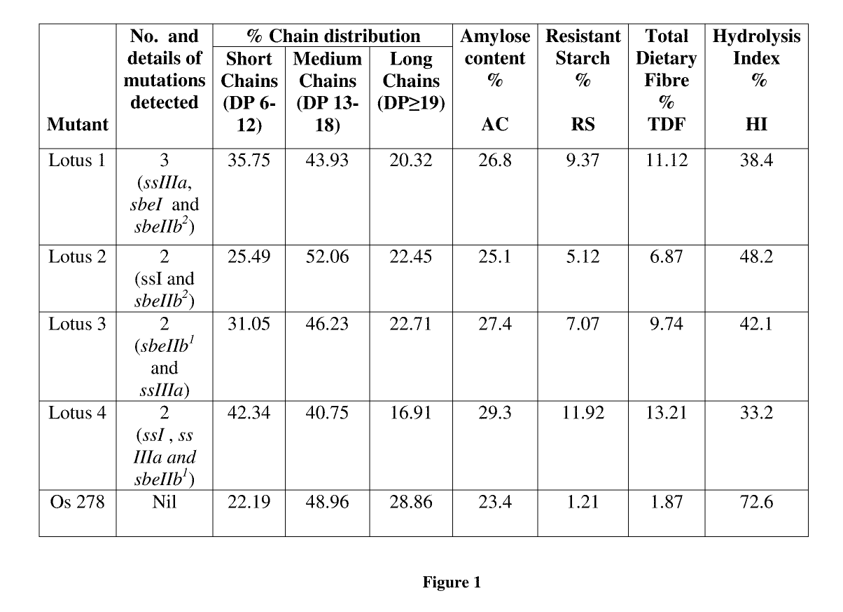

FIG. 1 shows a table depicting amylopectin chain distribution, amylose content, resistant starch content, total dietary fibre and hydrolysis index in the grains of the rice mutant lines Lotus 1-4 and wild type GFRL 78, in accordance to one or more embodiments of the invention. The amylose content is measured using a simplified I₂/KI assay. Resistant starch estimation is done using AOAC approved method 2002.02 with the kit of Megazyme International, Ireland.

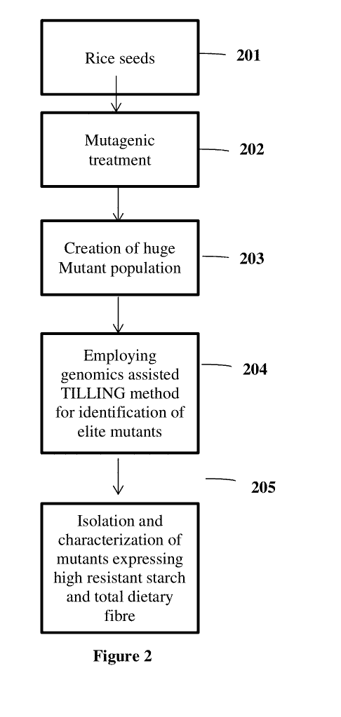

FIG. 2 illustrates a flowchart depicting a method of induction and screening mutation(s) in the genes encoding starch synthases and starch branching enzymes of a suitable rice variety in accordance with one or more embodiment of the present invention. As shown in FIG. 2, the seed of suitable rice variety is taken to perform mutation at step (201). At step (202), mutagenesis is performed by exposing seeds of a suitable rice variety with a mutagen that is ethyl methane sulfonate and or N—N-Nitroso Methyl Urea. At step (203), lots of mutants are produced by the mutation method. At step (204), Targeting Induced Local Lesions (TILLING) by sequencing (Tsai et al., 2011) is deployed to screen mutants with potential mutations that down-regulates key candidate genes coding for Starch Synthases and Starch Branching Enzymes. These mutations are then functionally validated for their role in down regulation of target genes through bioinformatic in silico tools SIFT (Ng and Henikoff, 2003) and Provean (Choi and Chan, 2015). Down regulation of such target enzymes leads to increased dietary fibre and resistant starch accumulation in rice grains. At step (205), the putative mutants selected are biochemically characterized for enhanced dietary fiber and resistant starch expression.

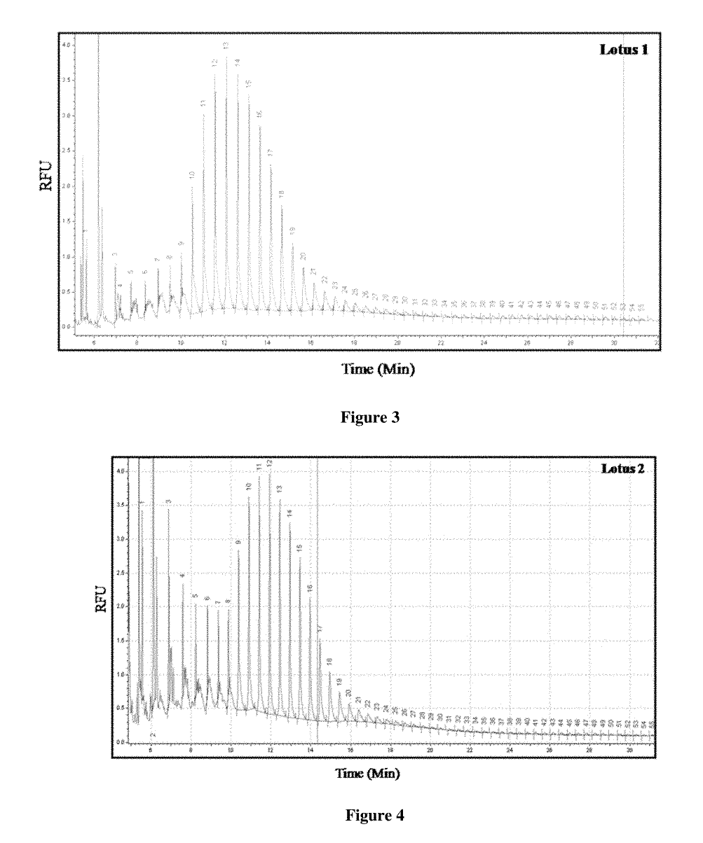

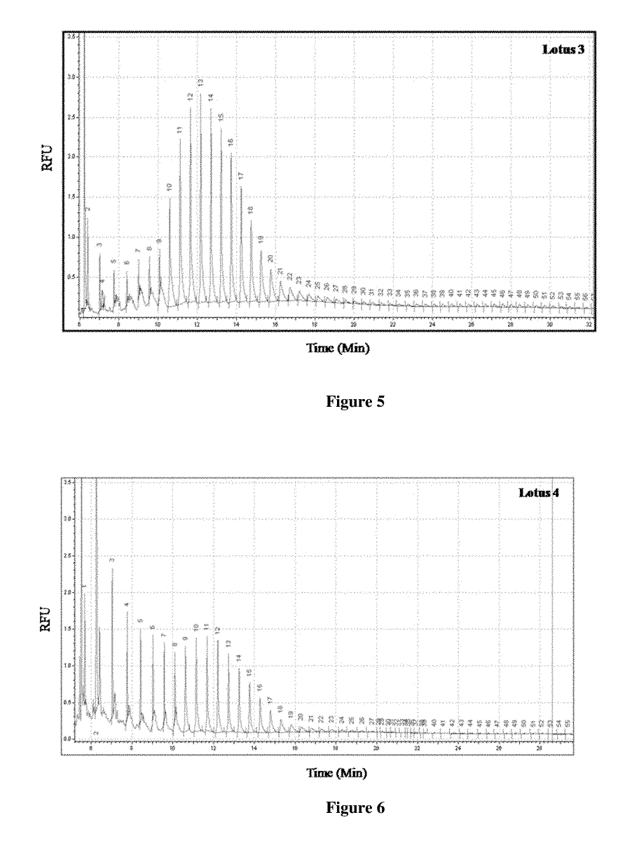

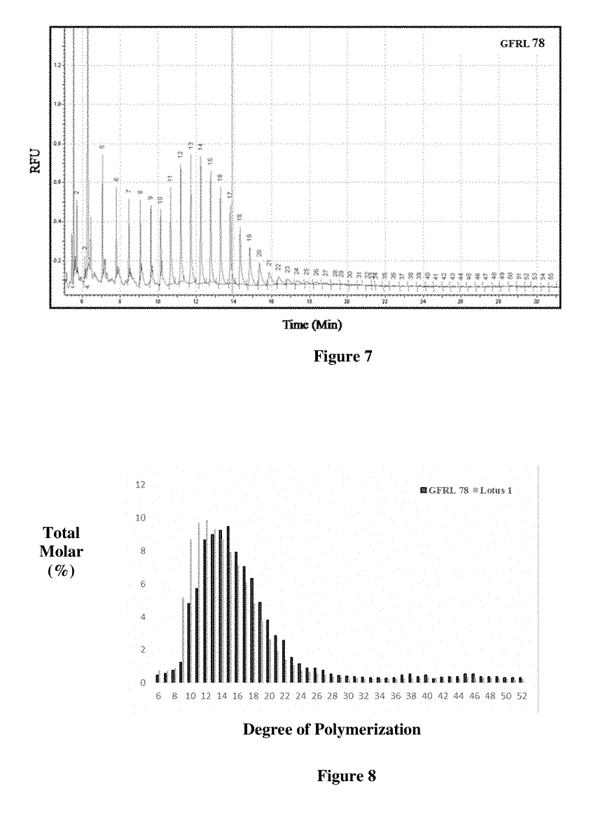

FIG. 3, FIG. 4, FIG. 5, FIG. 6 and FIG. 7 illustrate chromatograms generated from Fluorophore Assisted Capillary Electrophoresis (FACE). The graphs show that the proportion of amylopectin chains with lower chain length (DP 6 to 12) is predominant among all the mutants as compared to the wild type variety. Wild type variety exhibited higher proportion of moderate (DP 13-18) and longer (DP≥19) amylopectin chains. A general trend of chain length is evident in relationship to the mutations harbored by the mutants is that more the number of mutations harbored by a mutant higher will be the resistant starch and dietary fibre levels Among the mutants, the fourth mutant variety (Lotus 4), which is a triple mutant that harbors mutations in two genes coding starch synthases ssI and ssIIIa along with one mutation in a starch branching enzyme sbe IIb showed the highest proportion of short chains of 42.34% and all its biochemical parameters are most desirable with high values for AC (29.3%), RS (11.92%), and TDF (13.21%) and with lowest HI of 33.2%. It was followed by the first mutant variety (Lotus 1) with HI=35.75%, which harbored one starch synthase mutation (ssIIIa) and two starch branching mutations (sbeI and sbeIIb²). Irrespective of the number of mutations and the number of genes involved all mutants showed higher AC, RS, TDF and reduced HI as compared to wild type variety. As starch synthases SSIa and SSIIIa are postulated (Nakamura et al 2010) to play a role in the elongation of chain length of L type of amylopectin, which is commonly present in indica type of rice varieties their down regulation in the mutants leads to reduction in amylopectin chain length. While the mutations in Starch Branching Enzymes SBE IIa and SBEIIb and their down regulation has been proven to increase the amylose content in conjunction with reduction in amylopectin chain length in many cereals including rice (Nakamura et al 2003, Satoh et al 2003). The high level of amylose expression and reduced amylopectin chain length had both been postulated to result in the enhancement of Resistant Starch from moderate to high levels of 4 to 6% (Kawasaki et al 1993, Nishi et al 2001 and Fujita et al 2007). The double and triple mutants of this invention, where in mutations harbored in both gene families Starch Synthases and Starch Branching Enzymes together resulted in significant increase of Resistant Starch up to 11.92% and dietary fibre content up to 13.21%.

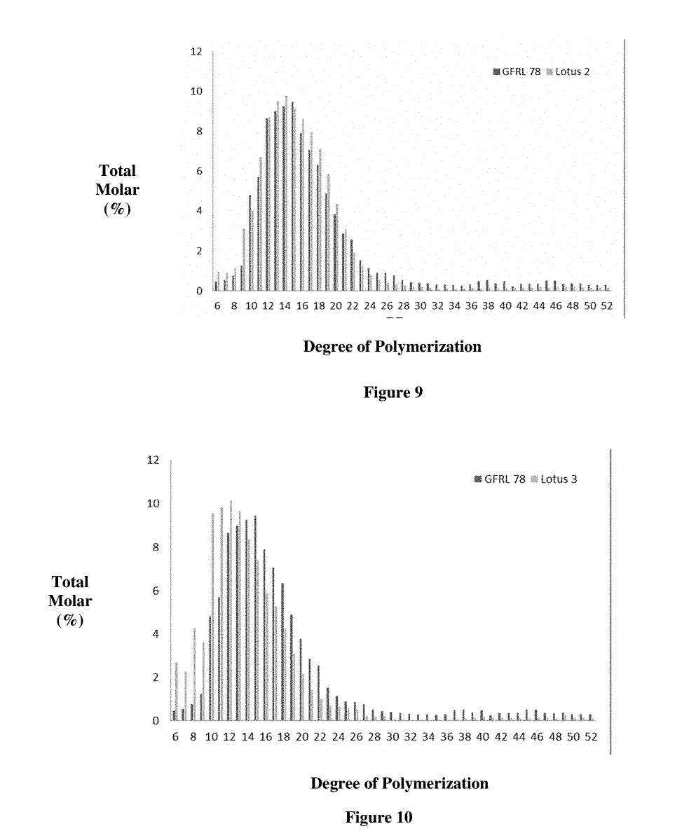

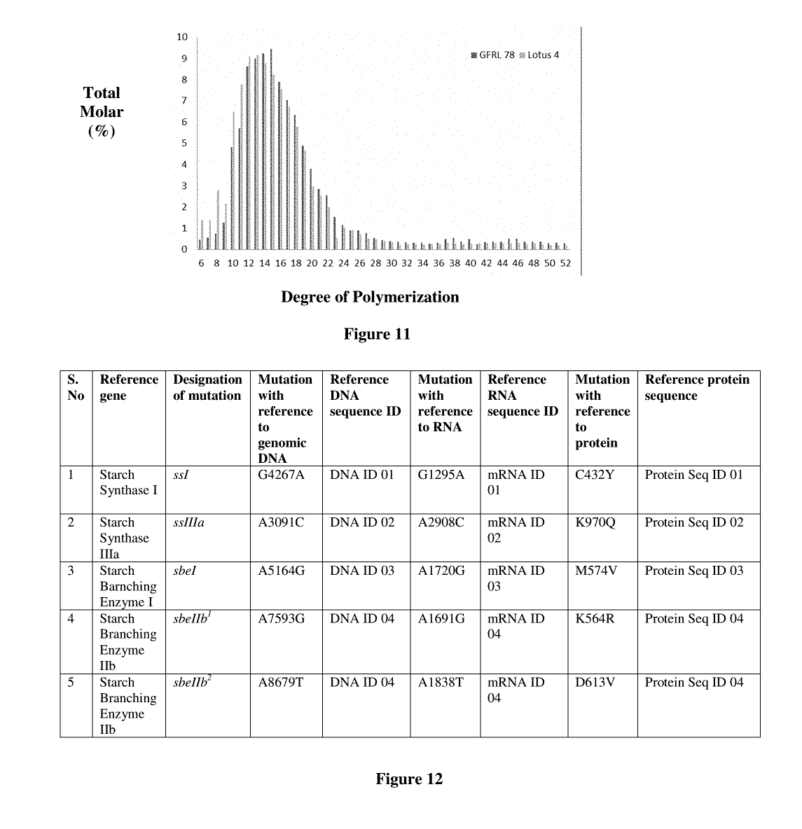

FIG. 8, FIG. 9, FIG. 10 and FIG. 11 shows graphs that compares amylopectin chain length distribution of Lotus 1, Lotus 2, Lotus 3 and Lotus 4 mutants with the wild type rice variety GFRL 78 in accordance to one or more embodiments of the present invention. The comparison of the data on chain length of amylopectin clearly indicates the preponderance of short chain amylopectin in mutants as compared to the wild type.

FIG. 12 shows a table depicting the list of mutations identified in the key candidate genes of mutant Lotus varieties, which are likely to increase the dietary fiber and resistant starch content in the endosperm, in accordance to one or more embodiments of the invention. The table shows the position of mutations, with respect to DNA, RNA and protein sequences.

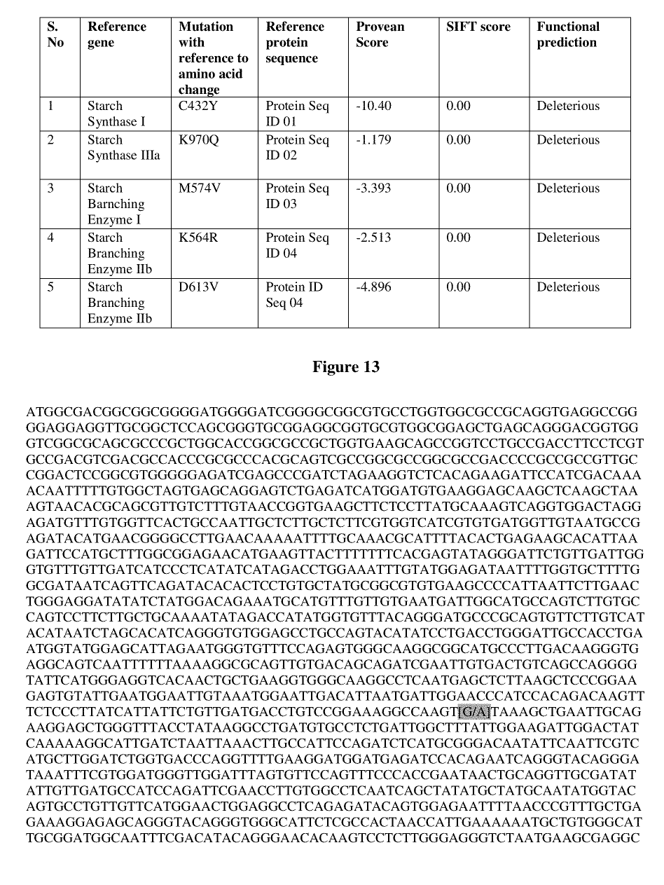

FIG. 13 shows a table depicting the list of mutations identified in the key candidate genes of mutant Lotus varieties, with reference to protein along with the reference protein sequence, Provean score, SIFT score, and functional prediction. Provean score of less than −1.3 is the thresh hold set to conclude an amino acid change is intolerable in that position of the polypeptide and hence the mutation is concluded as deleterious. SIFT predicts whether an amino acid substitution affects protein function. SIFT prediction is based on the degree of conservation of amino acid residues in sequence alignments derived from closely related sequences, collected through PSI-BLAST. SIFT is applied to naturally occurring nonsynonymous polymorphisms or laboratory-induced mis sense mutations. SIFT is a sequence homology-based tool that sorts intolerant from tolerant amino acid substitutions and predicts whether an amino acid substitution in a protein will have a phenotypic effect. SIFT score ranges from 0 to 1. The amino acid substitution is predicted damaging if the score is <=0.05, and tolerated if the score is >0.05.

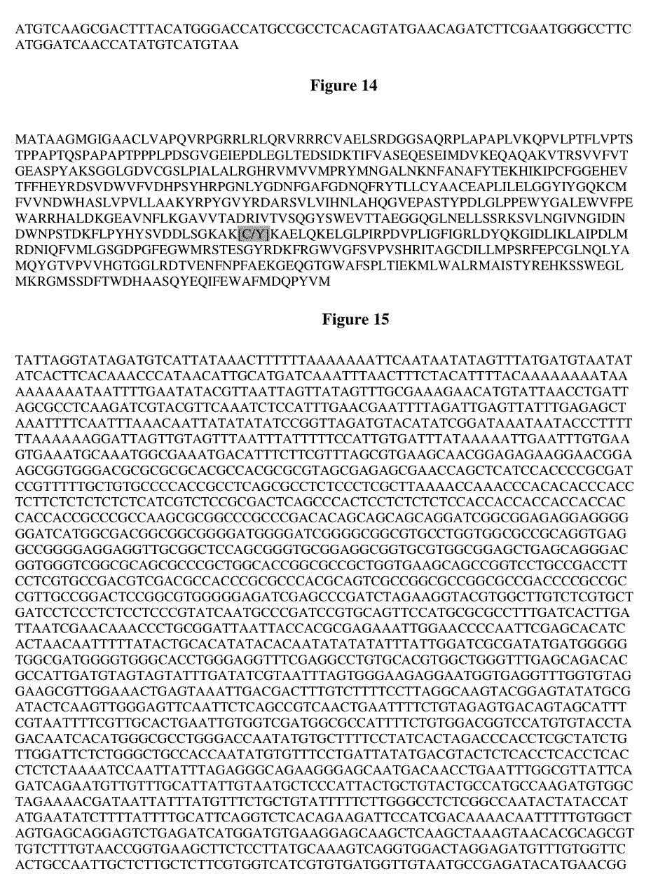

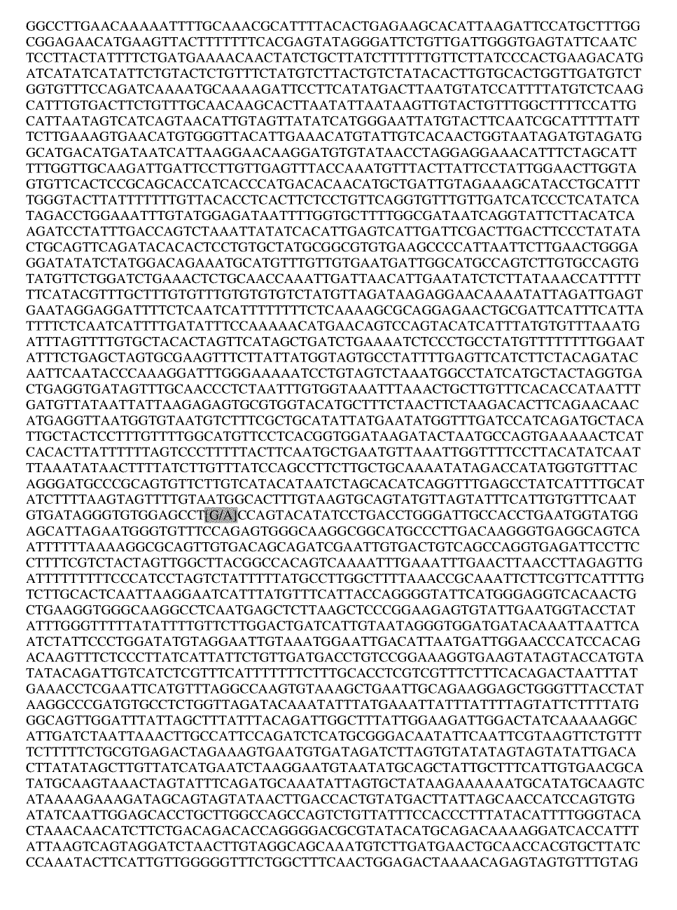

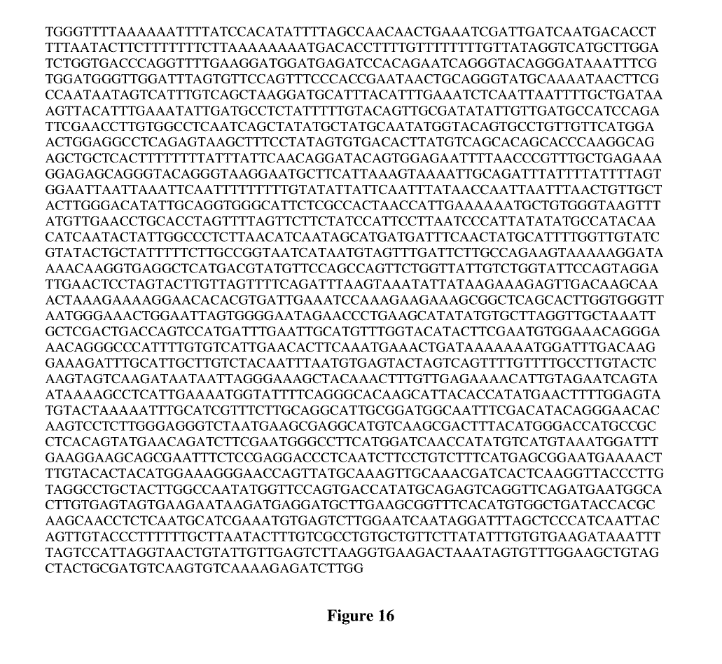

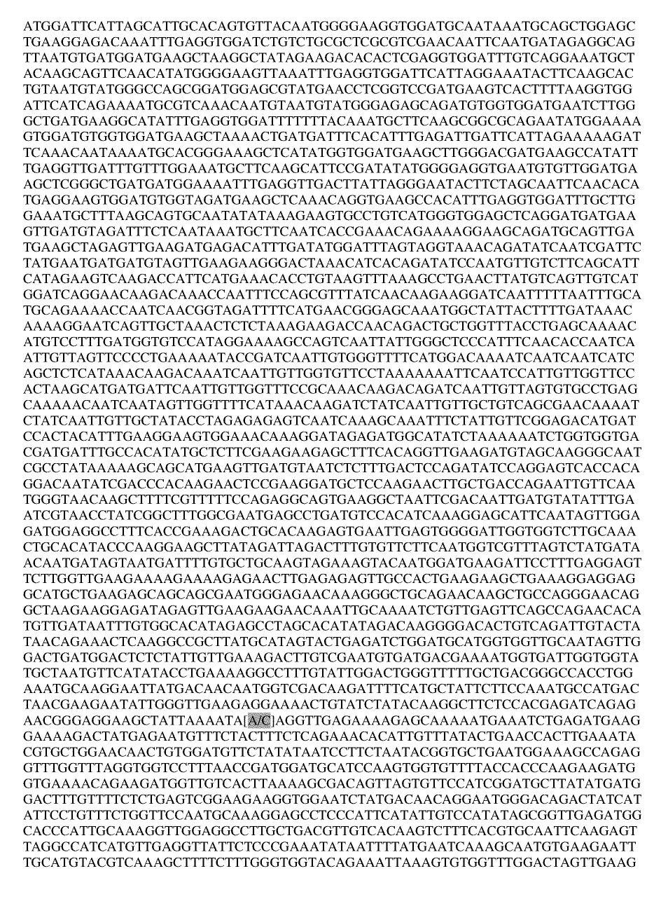

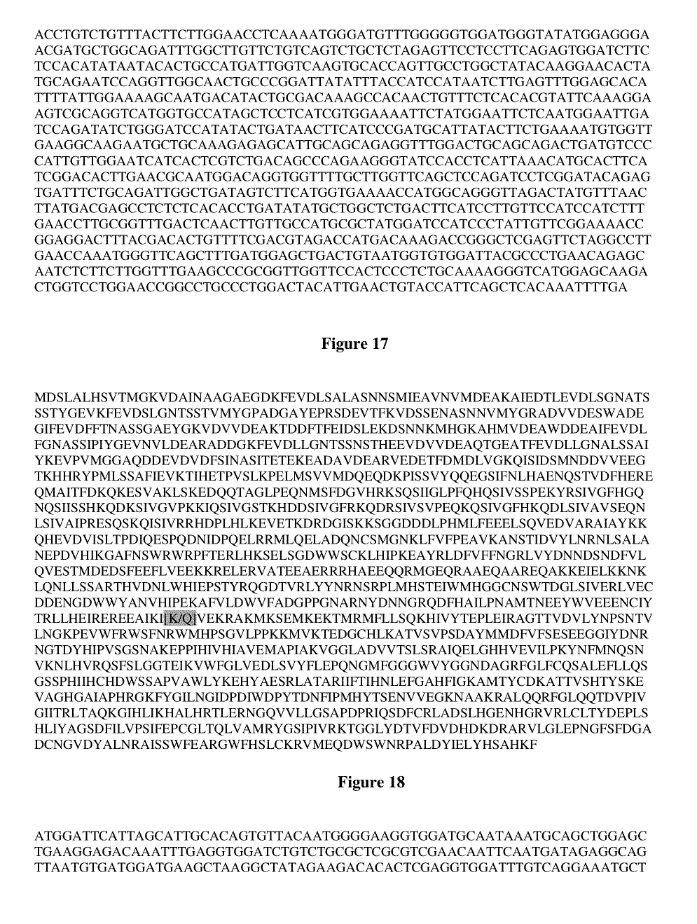

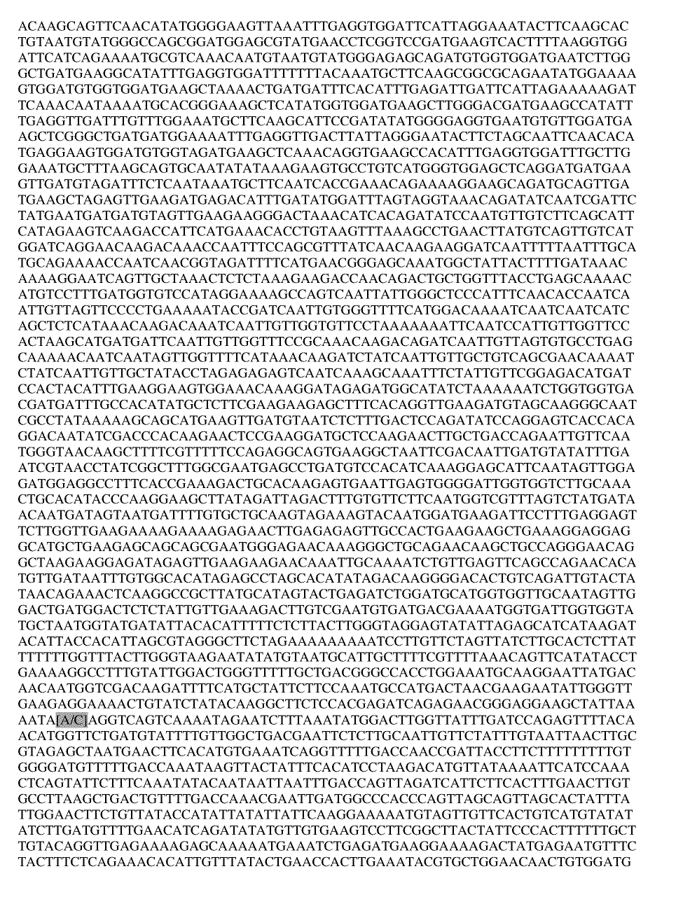

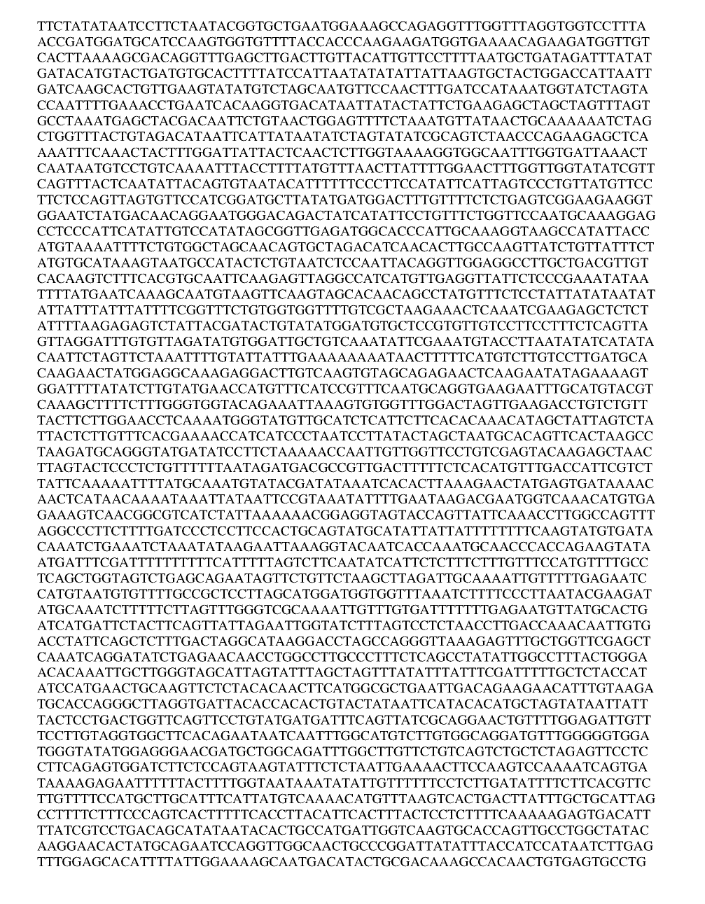

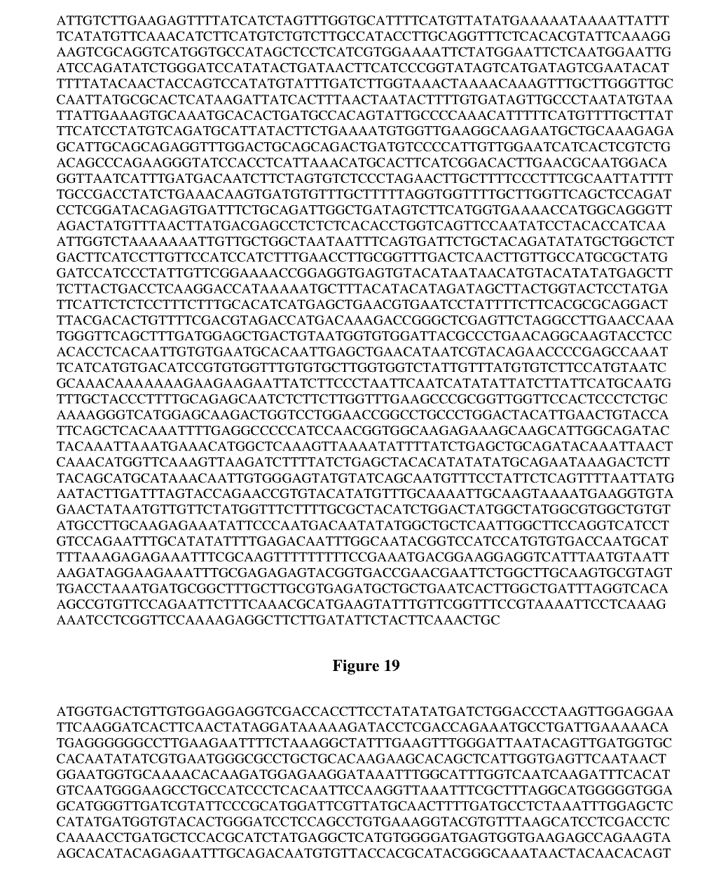

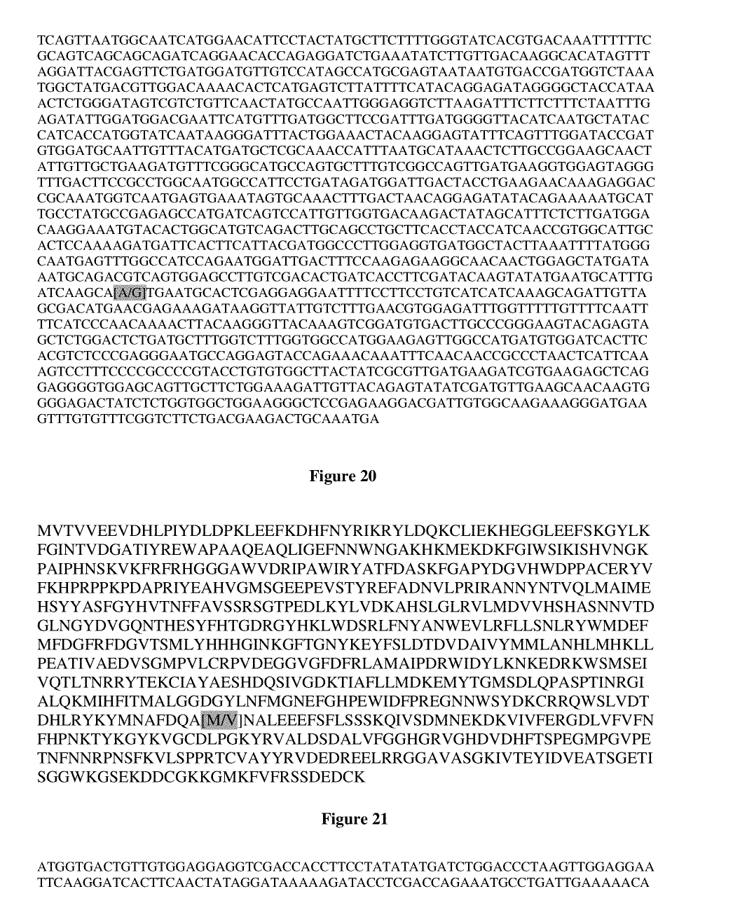

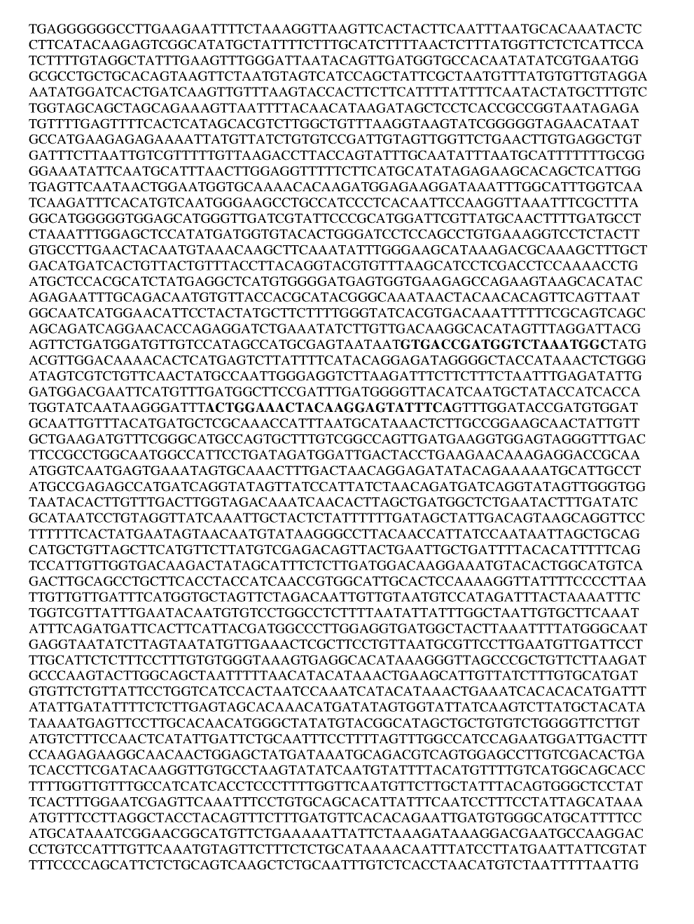

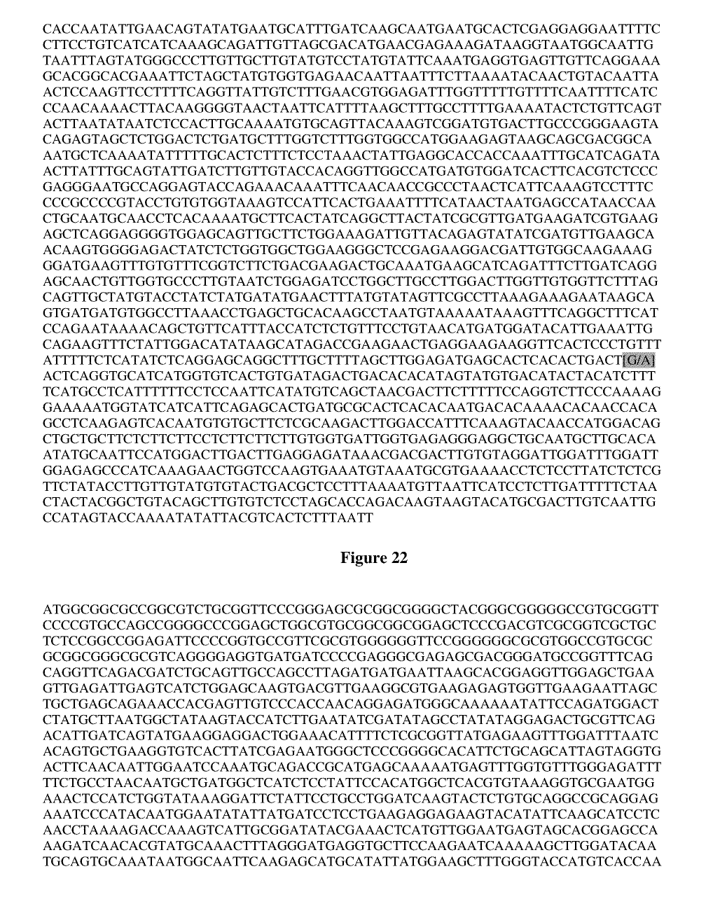

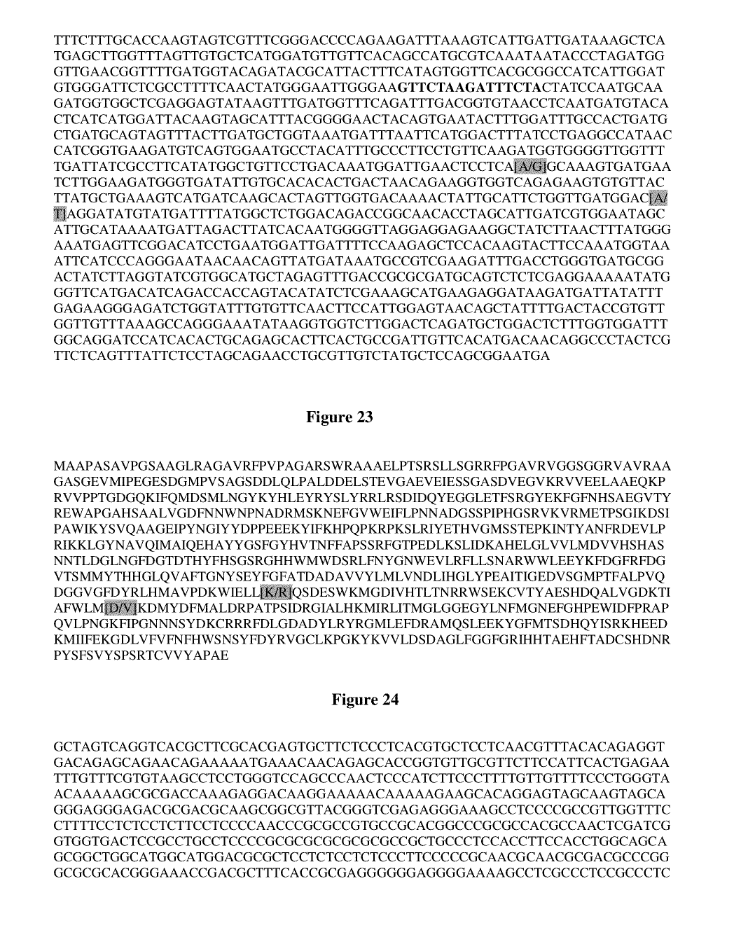

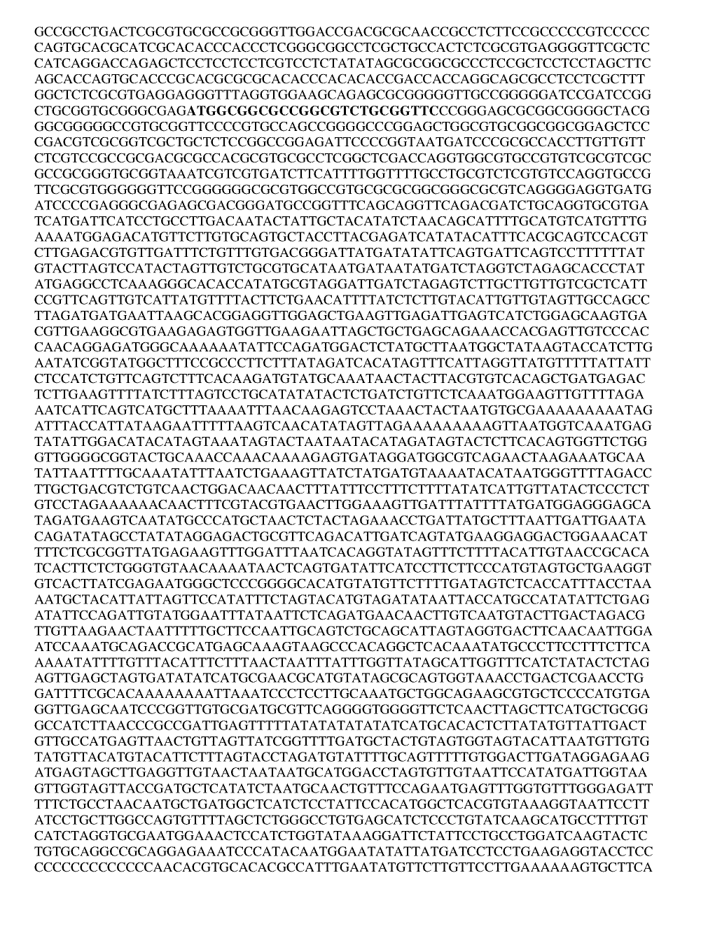

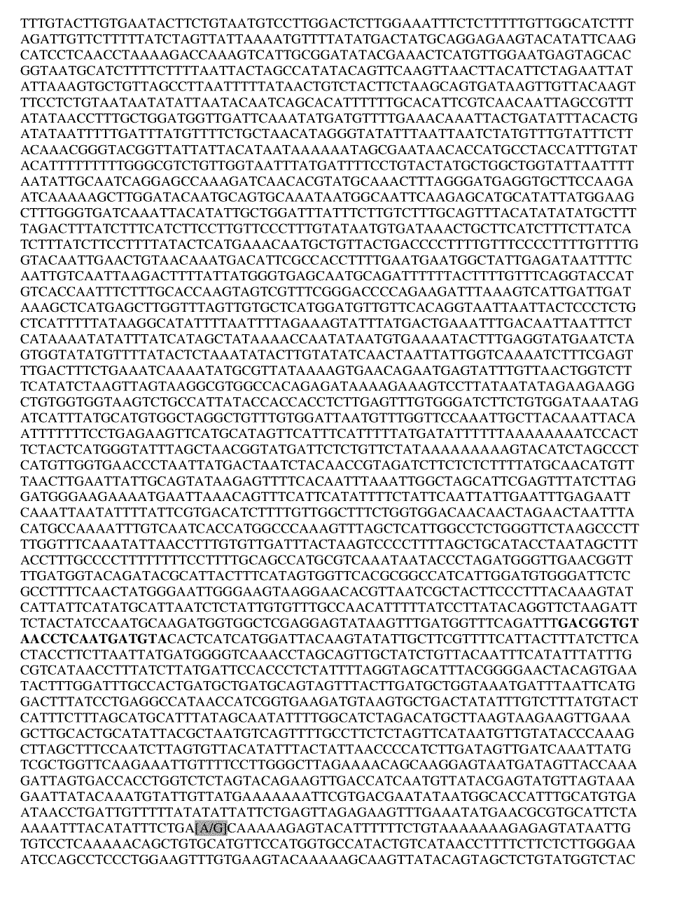

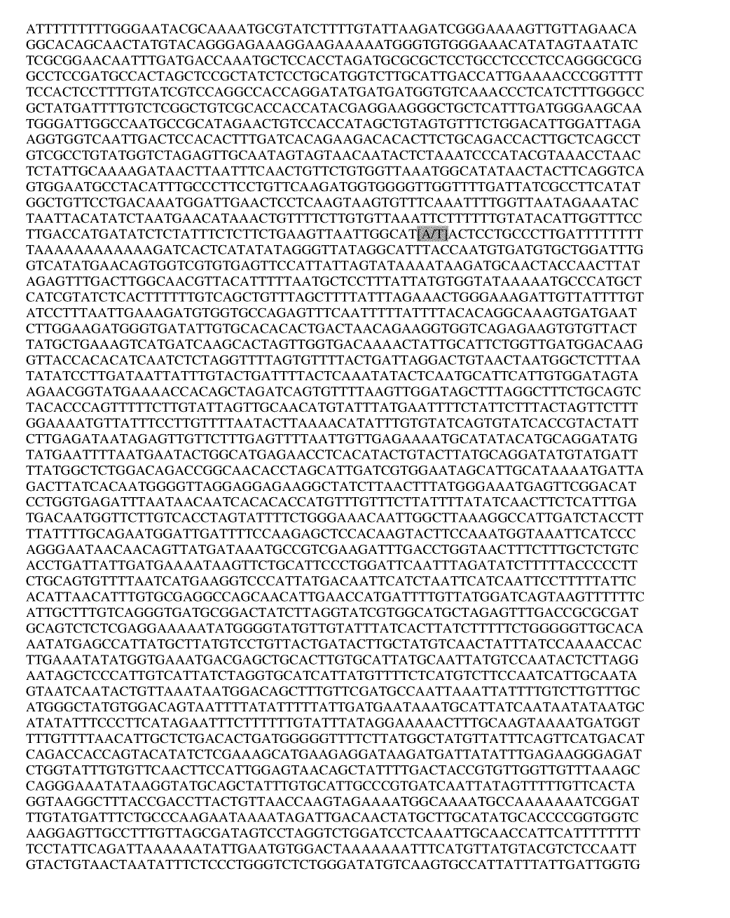

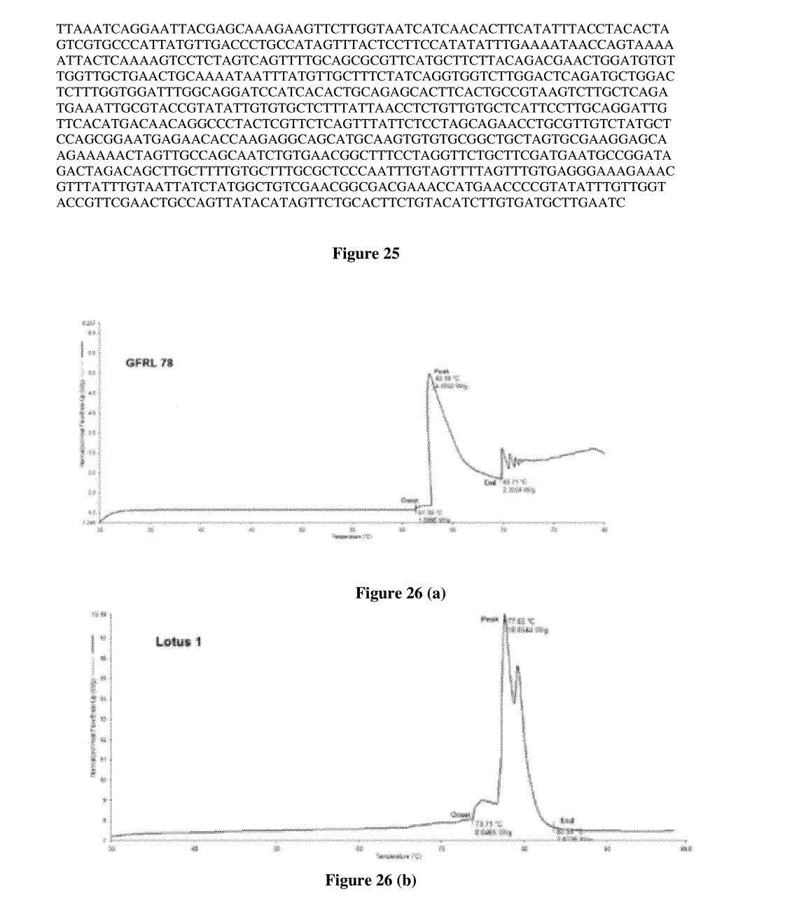

FIG. 14, FIG. 15, FIG. 16, FIG. 17, FIG. 18, FIG. 19, FIG. 20, FIG. 21, FIG. 22, FIG. 23, FIG. 24 and FIG. 25 show mRNA, protein and DNA sequence of Starch Synthase I, Starch Synthase Starch Branching enzyme I and Starch Branching enzyme IIb along with single, double or triple mutation, which is highlighted in the sequence.

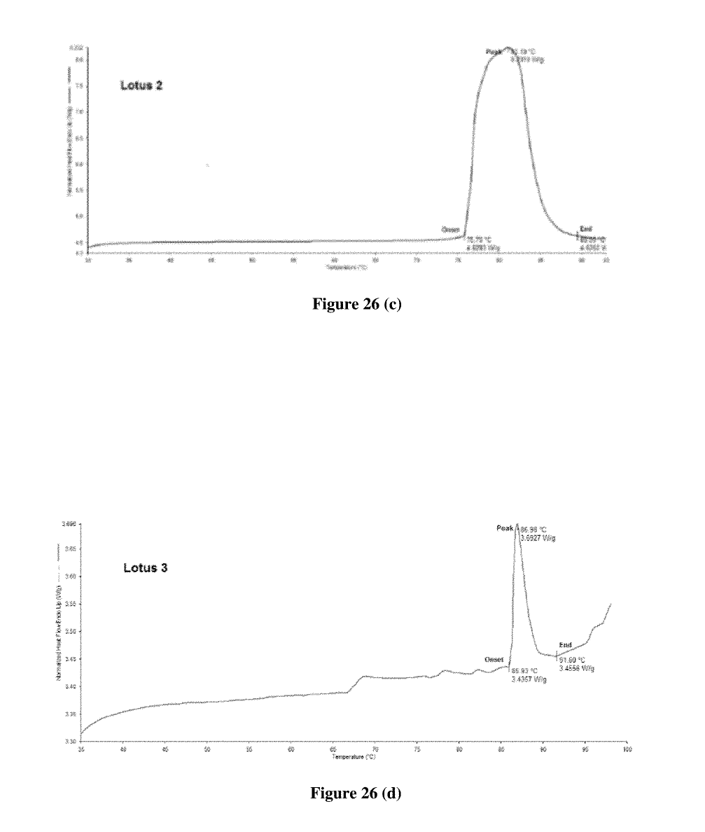

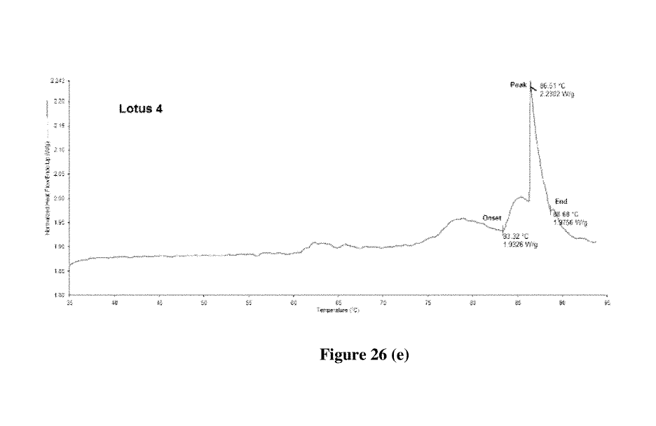

FIG. 26 illustrates the gelatinization properties of the starch from rice sample. The gelatinization and retrogradation properties of each rice sample are analyzed using a differential scanning calorimeter. The results showed that gelatinization of starch is a dynamic process during which starch in water undergoes a phase transition from solid to a viscous paste like state upon continuous heating. The gelatinization onset, peak and also the end point are dependent on temperature of water and the chemical composition of starch as well. FIG. 26(a-e) indicates the gelatinization profiles of the four mutants (Lotus 1 to 4) along with the wild type GFRL 78 respectively. It is observed that there is a significant increase in gelatinization temperature of 12° C. (Lotus 1) to 24° C. (Lotus 3) in mutants in comparison with wild type.

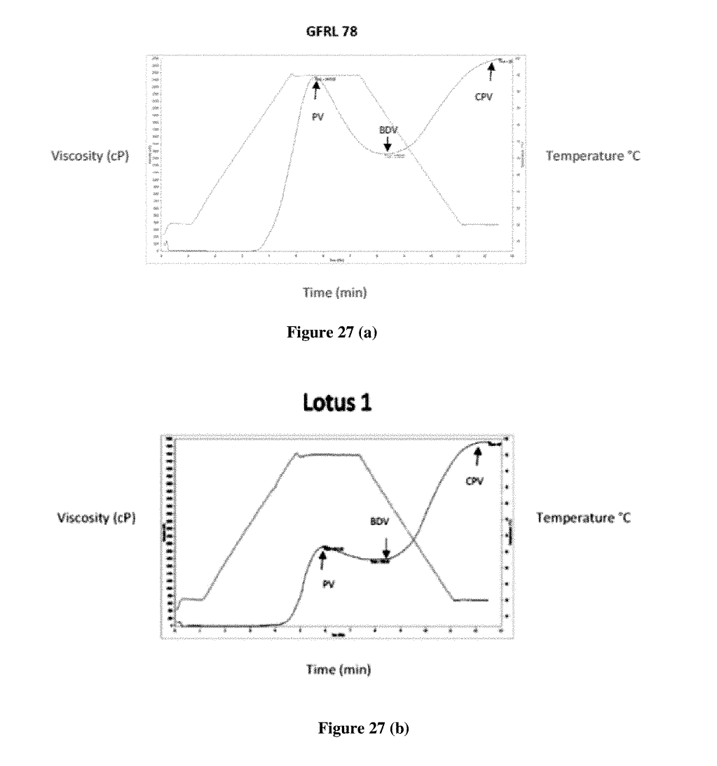

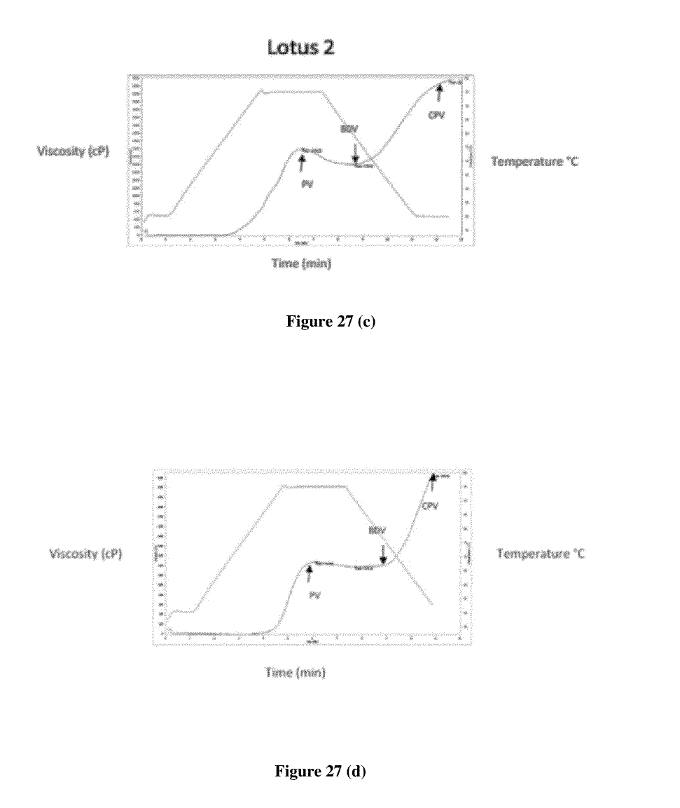

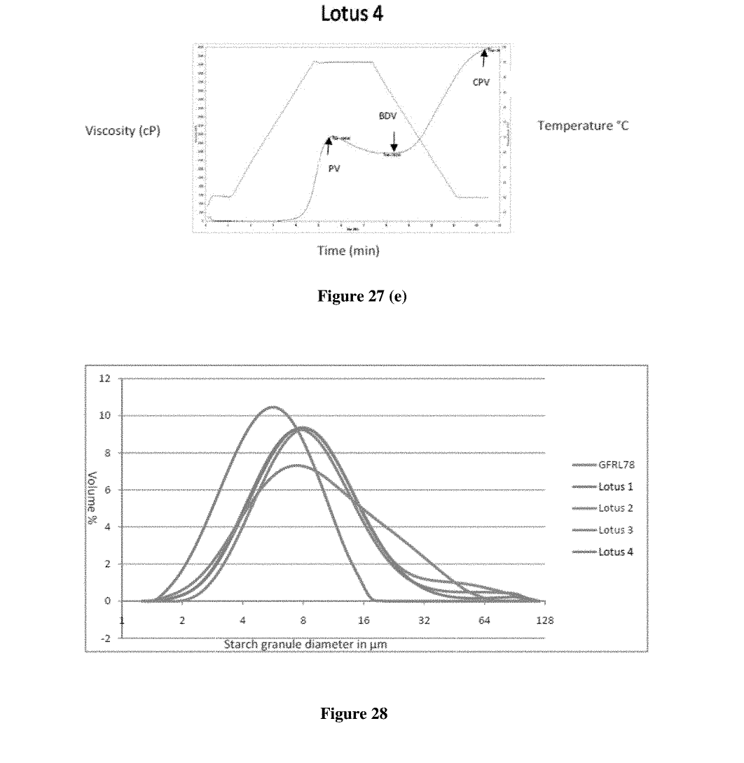

FIG. 27 illustrates the viscosity and pasting properties of starch from rice samples as determined by a Rapid Visco Analyser. The results shows that the viscosity and the pasting properties of starch dispersed in water and when measured under different temperature regimes (cold to hot and then back to cold conditions) give a clear indication about its chemical composition. FIG. 27(a-e) indicates the RVA results of the four mutant rice varieties Lotus 1 to Lotus 4 along with the wild type variety GFRL 78. It is evident that the peak viscosity (PV), Break Down Viscosity (BDV) and the final cool paste viscosities (CPV) indicate significantly lower values in all the four high RS mutants than the wild type.

FIG. 28 illustrates the granule size distribution of the starch of the rice. The graphical representation of the percentage proportion of volume occupied by starch granules of various sizes that the four mutants (Lotus 1 to 4) exhibited higher fractions of large size granules that their smaller counterparts as compared to the wild type GFRL 78. Many studies on characterization of particle size of various starches have indicated a negative correlation between granule size and resistant starch content. This has been attributed to the surface area and enzymatic interaction. Starch with more proportion of smaller granular composition exhibits larger surface area to interact with the enzyme and vice versa.

Having generally described this invention, a further understanding can be obtained by reference to certain specific examples, which are provided herein for purposes of illustration only and are not intended to be limiting unless otherwise specified.

### EXAMPLE 1

### RS Estimation Procedure

The RS content is estimated using the Megazyme kit. 100±1 mg of flour sample is taken in screw cap tubes in duplicates and gently tapped to ensure no sample adhered to the sides of the tube. Four ml of pancreatic α-amylase (3 Ceralpha Units/mg, 10 mg/ml) containing amyloglucosidase (AMG) (3 U ml⁻¹) was added to each tube. The tubes were tightly capped, dispersed thoroughly on a vortex mixer, and attached horizontally in a shaking water bath aligned in the direction of motion. The tubes are incubated at 37° C. with continuous shaking (200 strokes minute⁻¹) for 16 hr. After incubation, the tubes are treated with 4.0 ml of ethanol (99 percent) with vigorous mixing using a vortex mixer. After this, the tubes are centrifuged at 1,500×g (approx. 3,000 rpm) for 10 min (non-capped). The supernatant is carefully decanted and the pellet re-suspended in 8 ml of 50 percent ethanol. The tubes are again centrifuged at 1,500×g (approx. 3,000 rpm) for 10 min. Again, the supernatant is decanted and the suspension and centrifugation steps are repeated. The supernatant is decanted and the tubes inverted on absorbent paper to drain excess liquid. A magnetic stirrer bar (5×15 mm) is added to each tube, followed by 2 ml of 2 M KOH solution. The pellet is re-suspended (and the RS dissolved) by stirring for about 20 min in an ice or water bath over a magnetic stirrer. Then, 8 ml of 1.2 M sodium acetate buffer (pH 3.8) is added to each tube. Immediately, 0.1 ml of AMG (3300 U ml⁻¹) is added, the contents are mixed well under a magnetic stirrer, and the tubes are placed in a water bath at 50° C. The tubes are incubated for 30 minutes with intermittent mixing on a vortex mixer and are directly centrifuged at 1,500×g for 10 minutes. The final volume in each tube is approximately 10.3 (±0.05) ml. From each tube, 0.1 ml aliquot (in duplicate) of the supernatant was transferred into glass test tubes, added with 3.0 ml of GOPOD reagent, and mixed well using a vortex mixer. A reagent blank was prepared by mixing 0.1 ml of 0.1 M sodium acetate buffer (pH 4.5) and 3.0 ml of GOPOD reagent. Glucose standards are prepared by mixing 0.1 ml glucose (1 mg ml⁻¹) and 3.0 ml GOPOD reagent. The samples, blank and standards are incubated for 20 min at 50° C. The absorbance is measured at 510 nm against the reagent blank. Mega-Calc from Megazyme is used to calculate the RS content of the sample.

### EXAMPLE 2

### Degree of Polymerization of Amylopectin Chain

Pure starches are isolated from all the mutants and wild type and amylopectin chain length distributions of isolated starches are analyzed by Fluorophore Assisted Capillary Electrophoresis (FACE). The isolated starches are debranched (at 37° C. for 2 h) using iso-amylase enzyme (10 U) and labeled with 1-Aminopyrene-3,6,8-Trisulfonic Acid (APTS). FACE is conducted using the P/ACE System 5010, which is equipped with a 488 nm laser module. The N—CHO (PVA) capillary with a preburned window is used for separation of debranched samples. Maltose is used as an internal standard. Separation is conducted at 10° C. for 30 min. The degree of polymerization (DP) is allocated to peaks based on the migration time of maltose.

### EXAMPLE 3

### Assessment of Gelatinization Temperature

Gelatinization and retrogradation properties of each rice sample are analyzed using a differential scanning calorimeter, DSC6000 (Perkin Elmer, USA). To investigate the thermal properties in a 50 μl aluminum pan, 15 mg of the flour sample obtained from polished raw rice samples of mutants Lotus 1 to 4 and the control variety GFRL 78 are added, combined with 35 μL of deionized water and the sample concentration is adjusted to 30%. As a reference, 50 μL deionized water is added and adjusted to an equal weight. Regarding the measurement condition, the temperature is increased from 30° C. to 100° C. at the rate of 3° C./min. The analytical properties measured are gelatinization start, peak, and end temperatures (To, Tp, and Te, respectively).

### EXAMPLE 4

### Viscosity and Pasting Property Assessment

The rice samples are milled and grinded using the method described previously. Paste viscosity is determined on a Rapid Visco Analyzer (RVA) instrument using the American Association of Cereal Chemistry (AACC) (1995) Standard Method 61-02. The RVA 4500 model is used (Perten Instruments, Sweden). The RVA uses 3 g of rice flour in 25 ml water (Juliano, 1996). The temperature is set at 50° C. for 1 minute, heating to 95° C. at 12° C. per minute and 2.5 minutes at 95° C. The cooling is 50° C. at 12° C. per minute. The heating is at 50° C. for 54 seconds for a total running time of 12.5 minutes. The RVA breakdown, the consistency and the setback at 50° C. and 30° C. is calculated. The units for all the calculated parameters are in Rapid Visco Units (RVU). One unit RVU=10 cp. The viscosity characteristics obtained from the RVA can be described by three important parameters: the peak (first peak viscosity after gelatinization), hot paste (paste viscosity at the end of 95° C. holding period), and cool paste viscosity (paste viscosity at the end of the test). Breakdown is derived from peak minus hot paste viscosity, setback is derived from cool paste viscosity minus peak viscosity values, consistency Viscosity is derived from cool paste viscosity minus hot paste viscosity. The different parameters obtained are measured in Rapid Visco Units (RVU).

### EXAMPLE 5

### Starch Granule Size Distribution Analysis

Starch Extraction

Starch extraction is carried out as described by Lumdubwong and Seib (2000) with some modification. The ground rice meal (1 g) is steeped overnight with 0.01M NaOH (5 mL) and 100 μL of 1% protease at 37° C., and neutralized using 1M HCl. The solution is centrifuged at 3,000 g and the supernatant is discarded. The precipitate is suspended in water (1 mL), layered over 80% (w/v) Cesium Chloride solution (1 mL) and centrifuged at 13,000 rpm for 20 min. The pellet obtained is suspended with water and filtered through 100 μm pore size nylon filter. Supernatant is discarded and dark tailing layer is removed with spatula. The starch pellet is washed thrice with 1 mL of water and centrifuged at 13,000 rpm for 10 min, followed by acetone (1 mL) and centrifuged at 13,000 rpm for 10 min and finally air dried overnight.

Starch Granule Size Distribution

Starch granule size distribution of the extracted starch is determined by laser diffraction technique using particle size analyzer (Mastersizer 2000, Malvern Instruments, Malvern, England). The pure starch (30 mg) is weighed and dispersed in 1 ml of 1% Sodium dodecyl sulfate (SDS; Fisher scientific, USA). About 200 μl of starch slurry was used for size analysis at a pump speed of 1700 rpm (Asare et al., 2011).

While at least one exemplary embodiment has been presented in the foregoing summary and detailed description, it should be appreciated that a vast number of variations exist. It should also be appreciated that the exemplary embodiment or exemplary embodiments are only examples, and are not intended to limit the scope, applicability, or configuration in any way. Rather, the foregoing summary and detailed description will provide those skilled in the art with a convenient road map for implementing an exemplary embodiment, it being understood that various changes may be made in the function and arrangement of elements described in an exemplary embodiment without departing from the scope as set forth in the appended claims and their legal equivalents.

We claim:

1. A rice plant comprising one or more mutations in a combination of two, three or four genes that includes SSI, SS IIIa, SBE I and SBE IIb; wherein said rice plant produces seed that germinates, and further wherein grain from said rice plant has an increased resistant starch or total dietary fibre level as compared to grain from a wild type rice plant.

2. The rice plant of claim 1, further comprising a reduced levels of enzymes Starch Synthase I and/or Starch Synthase IIIa and in combination with reduced levels of Starch Branching Enzyme I and/or Starch Branching Enzyme IIb in starch granules resulting from mutations in a combination of two, three or four genes coding these enzymes of said plant as compared to starch granules of a wild type rice plant.

3. The rice plant of claim 1, wherein starch of the grain has an enhanced amylose content of more than 26% as compared to the grains of the wild type rice plant.

4. The rice plant of claim 1, wherein starch in the grains has an enhanced resistant starch content of more than 6% as compared to the grains of wild type rice plant.

5. The rice plant of claim 1 which is Oryza sativa of race indica type.

6. Rice grain from the rice plant of claim 1.

7. Flour comprising a cell of the rice grain of claim 1.

8. A food or beverage product comprising a cell of the rice plant of claim 1.

9. A rice seed, pollen grains, plant parts or progenies derived in any form from the rice plant of claim 1 either through plant breeding or molecular breeding or any biotechnological approaches thereof.
